(19) United States
(12) Patent Application Publication
(10) Pub. No.: US 2013/0253313 A1
(43) Pub. Date: Sep. 26, 2013

(54) AUTOFOCUSING ENDOSCOPE AND SYSTEM

(71) Applicants: Jin Kang, Ellicott City, MD (US); Marcin Arkadiusz Balicki, Baltimore, MD (US); Rajesh Kumar, Hunt Valley, MD (US); Russell H. Taylor, Severna Park, MD (US)

(72) Inventors: Jin Kang, Ellicott City, MD (US); Marcin Arkadiusz Balicki, Baltimore, MD (US); Rajesh Kumar, Hunt Valley, MD (US); Russell H. Taylor, Severna Park, MD (US)

(73) Assignee: The Johns Hopkins University, Baltimore, MD (US)

(21) Appl. No.: 13/813,896

(22) PCT Filed: Aug. 2, 2011
(86) PCT No.: PCT/US11/46242

§ 371 Date: Jun. 11, 2013

Related U.S. Application Data
(60) Provisional application No. 61/370,044, filed on Aug. 2, 2010.

(51) Int. Cl. A61B 1/00; A61B 3/10; A61B 1/07

(52) U.S. Cl. CPC A61B 1/00188; A61B 1/00165; A61B 1/07; A61B 3/102; A61B 1/00004
USPC 600/425; 600/167

(57) ABSTRACT

An autofocusing endoscope includes an objective lens, a relay optical system arranged to relay an image between the objective lens and a proximal end of the autofocusing endoscope, an optical fiber arranged with a distal end proximate the objective lens, a light source arranged to couple light into the optical fiber, an optical detection system arranged to receive and detect light from the optical fiber, and a data processor constructed to communicate with the optical detection system while in operation. The data processor is configured to determine a distance of a surface to be imaged through the objective lens and provide instructions for adjusting a focus of the autofocusing endoscope of the surface.

### CROSS-REFERENCE OF RELATED APPLICATION

This application claims priority to U.S. Provisional Application No. 61/370,044 filed Aug. 2, 2010, the entire contents of which are hereby incorporated by reference.

This invention was made with Government support of Grant No. 1R01 EB 007969-01, awarded by the Department of Health and Human Services, The National Institutes of Health (NIH); and Grant No. EEC-9731478, awarded by the National Science Foundation (NSF). The U.S. Government has certain rights in this invention.

### BACKGROUND

1. Field of Invention

The field of the currently claimed embodiments of this invention relates to endoscopes and systems, and more particularly to autofocusing endoscopes and systems.

2. Discussion of Related Art

In the case of eye surgery, current stereomicroscopes provide suboptimal resolution along with limited field of view, especially of peripheral structures of the retina. Further, stereomicroscopes do not provide any visibility of tissue beyond the top layer. GRIN lens micro-endoscopes can provide high resolution but suffer from very shallow depth of field, and a very narrow field of view, making them impractical for handheld applications. In vitreoretinal surgery a handheld endoscopic imager may be used in difficult cases where the damaged cornea does not permit the direct use of the ophthalmoscope. It can also be used for fine intraocular diagnostic purposes where high resolution real-time imagery is used to explore the surface of the retina. In addition, the fusion of cross-sectional sample information with correlated on-face imaging can provide the surgeon with valuable information about the state of the underlying tissue. There thus remains a need for improved endoscopes and systems that include the endoscopes.

### SUMMARY

An autofocusing endoscope according to an embodiment of the current invention includes an objective lens, a relay optical system arranged to relay an image between the objective lens and a proximal end of the autofocusing endoscope, an optical fiber arranged with a distal end proximate the objective lens, a light source arranged to couple light into the optical fiber, an optical detection system arranged to receive and detect light from the optical fiber, and a data processor constructed to communicate with the optical detection system while in operation. The data processor is configured to determine a distance of a surface to be imaged through the objective lens and provide instructions for adjusting a focus of the autofocusing endoscope of the surface.

### BRIEF DESCRIPTION OF THE DRAWINGS

Further objectives and advantages will become apparent from a consideration of the description, drawings, and examples.

FIG. 1 is a schematic illustration of an autofocusing endoscope according to an embodiment of the current invention.

FIG. 2 is a schematic illustration of a portion of a hand-held autofocusing endoscope according to an embodiment of the current invention.

FIG. 3 is a schematic illustration of a probe end of an autofocusing endoscope according to an embodiment of the current invention in which an optical fiber for an OCT system is integrated into a bundle of optical fibers of a relay optical system.

FIG. 4 is a schematic illustration of a probe end of an autofocusing endoscope according to an embodiment of the current invention in which an optical fiber for an OCT system is arranged alongside a bundle of optical fibers of a relay optical system.

FIG. 5 is a schematic illustration of a probe end of an autofocusing endoscope according to an embodiment of the current invention in which a laser spot on, and an imaging area of, a surface are shown.

FIG. 6 is a schematic illustration of an autofocusing endoscope according to an embodiment of the current invention in which the autofocusing endoscope is integrated into a robotic system.

FIG. 7 illustrates two examples of possible mosaic imaging patterns according to some embodiments of the current invention.

FIG. 8 illustrates an example of an image mosaic, an OCT path and an OCT cross section according to an embodiment of the current invention.

FIG. 9 illustrates two additional embodiments of the current invention that include a scanned OCT system. In the upper embodiment, optical fibers are selectively optically coupled by the scanning device.

FIG. 10 shows a prototype of a hand-held autofocusing endoscope according to an embodiment of the current invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

The term “light” as used herein is intended to have a broad meaning that can include both visible and non-visible regions of the electromagnetic spectrum. For example, visible, near infrared, infrared and ultraviolet light are all considered as being within the broad definition of the term “light.”

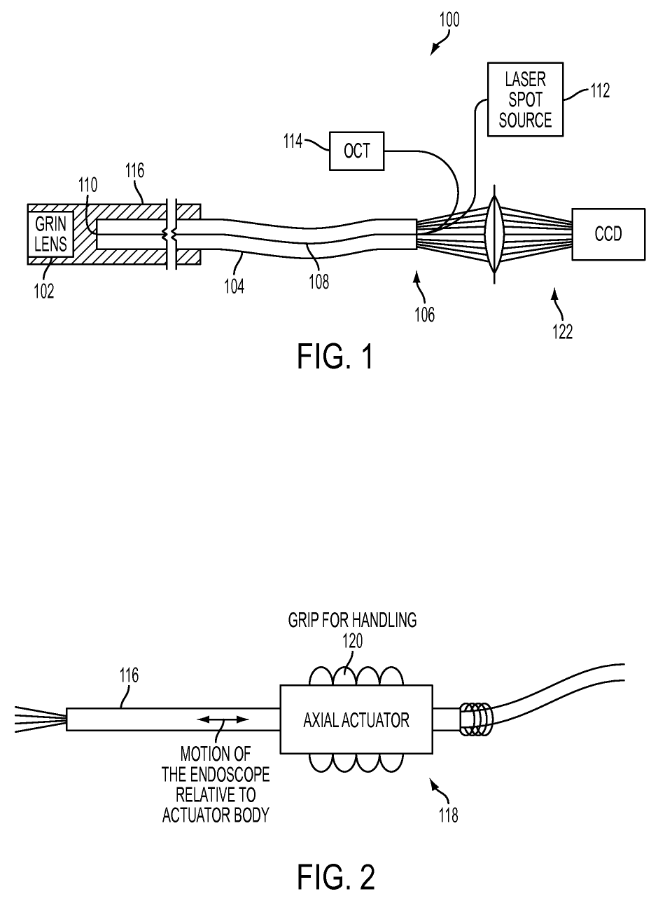

FIG. 1 provides a schematic illustration of an autofocusing endoscope 100 according to an embodiment of the current invention. The autofocusing endoscope 100 has an objective lens 102, a relay optical system 104 arranged to relay an image between the objective lens 102 and a proximal end 106 of the autofocusing endoscope 100, an optical fiber 108 arranged with a distal end 110 proximate the objective lens 102, a light source 112 arranged to couple light into the optical fiber 108, and an optical detection system 114 arranged to receive and detect light from the optical fiber 108. The autofocusing endoscope 100 also has a data processor constructed to communicate with the optical detection system 114 while in operation. The data processor can be combined together with the optical detection system or could be one or more separate components according to some embodiments of the current invention. The data processor is configured to determine a distance of a surface to be imaged through the objective lens 102 and provide instructions for adjusting a focus of the autofocusing endoscope 100 of the surface. The term “surface” can refer to any portion of a surface of an object being imaged.

The autofocusing endoscope 100 also has an endoscope body 116 and an actuator assembly 118 (FIG. 2) attached to the endoscope body 116 such that the actuator assembly 118 moves at least the objective lens 102 and the distal end 110 of the optical fiber 108 relative to the surface based on instructions received from the data processor to adjust the focus. As is illustrated in the embodiment of FIG. 2, the autofocusing endoscope 100 is a hand-held autofocusing endoscope and the actuator assembly 118 can have a hand grip 120, for example. In some embodiments, it has been found suitable to use a piezoelectric micro-motor (LEGS-L01S-11, PiezoMotor AB, Sweden) in the actuator assembly 118. However, the general concepts of the current invention are not limited to this particular example.

In some embodiments, the objective lens 102 can be a gradient index (GRIN) objective lens. In some embodiments, the objective lens 102 can include at least one of a compound lens, a refractive lens, a diffractive lens, or a gradient index (GRIN) lens, for example. In some embodiments, the relay optical system 104 can be a bundle of optical fibers. In some embodiments, the relay optical system 104 can be a lens system. In some embodiments, the relay optical system 104 can include at least one of a refractive lens, a diffractive lens, a GRIN lens, an optical fiber, a light pipe, or an optical waveguide, for example.

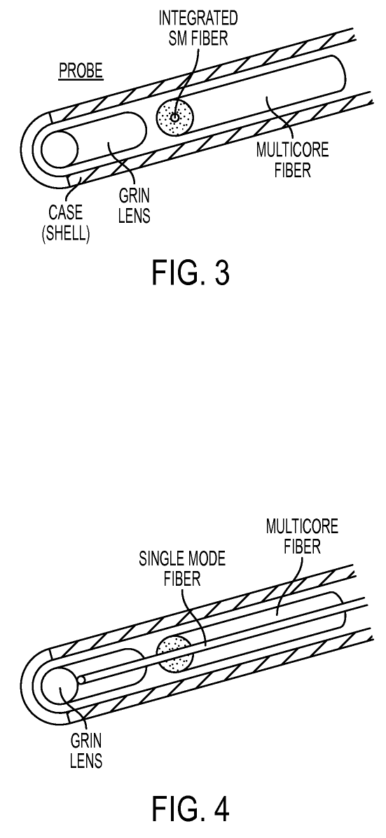

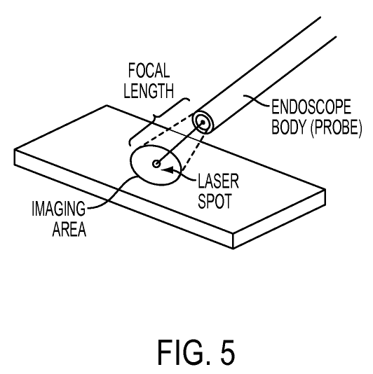

In some embodiments, the relay optical system 104 can be a bundle of optical fibers and the optical fiber 108 can be combined into a bundle with the bundle of optical fibers of the relay optical system 104 such that the optical fiber 108 emits and receives light from the distal end 110 through the objective lens 102 (See also FIGS. 3-5). Some or all of the optical fibers can be single mode optical fibers in some embodiments of the current invention. In an embodiment, the optical fiber 108 can be a single mode optical fiber and the optical fibers of the bundle of optical fibers of the relay optical system 104 can be multimode optical fibers, for example.

In some embodiments, the optical fiber 108, the optical detection system 114, the light source 112 and the data processor together form an optical coherence tomography system (OCT). In some embodiments, the OCT system can be a common path OCT system in which the OCT system has measurement and reference arms that coincide within the optical fiber 108. In some embodiments, the OCT system can be a Fourier domain OCT system (FD-OCT). However, the broad concepts of the current invention are not limited to only FD-OCT systems. For example, time domain OCT systems could be used in some embodiments. In addition, the broad concepts of the current invention are not limited to only autofocusing endoscopes that have an integrated optical coherence tomography system. For example, other interferometric and/or range-determination systems may be incorporated within the autofocusing endoscope according to other embodiments of the current invention.

In some embodiments of the current invention, the autofocusing endoscope 100 also has an illumination light source 122 optically coupled to the relay optical system 104 to provide illumination light to illuminate the surface being imaged. The illumination source can be, but is not limited to, a white light source for example. The autofocusing endoscope 100 can be used for direct observation by a user, or it can include an image pickup system to display and/or record images.

In operation, a user holds the autofocusing endoscope 100 by hand grip 120. The OCT system in this embodiment permits the detection and determination of the distance to the region of the surface at which the light from the OCT system is directed. In this embodiment, the light from the OCT system passes through the objective lens 102; however, the distal end 110 of the optical fiber 108 could alternatively be arranged such that it is fixed alongside the objective lens 102, for example. In either case, there is a fixed spatial relationship between the position of the distal end 110 of the optical fiber 108 and the position of the objective lens. By determining the distance the objective lens 102 is from the object (surface, etc.) being imaged, and knowing the desired distance for good focus, the data processor provides signals for the actuator assembly 118 to move the body 116 of the autofocusing endoscope 100 towards or away from the object being imaged if a correction in focus is needed. The fact that the actuator assembly 118 is arranged at a proximal portion in this embodiment allows the distal end to remain small and compact and can be free of electrical components, if desired.

In some embodiments, the illumination light can be coupled into the bundle of optical fibers by a fiber coupler or beam splitter, for example. A part of the bundle of optical fibers can be used as a light source (i.e., outer ring of the imaging bundle) while the rest of the bundle will be used to collect the image, for example. Illumination can also be introduced by a light probe mounted in parallel with the fiberscope according to some embodiments of the current invention.

In an alternative embodiment that does not use an OCT system, a narrow band such as provided by a laser, for example, can project a spot of light through the objective lens. A minimum spot size, for example, would then correspond to a good focus.

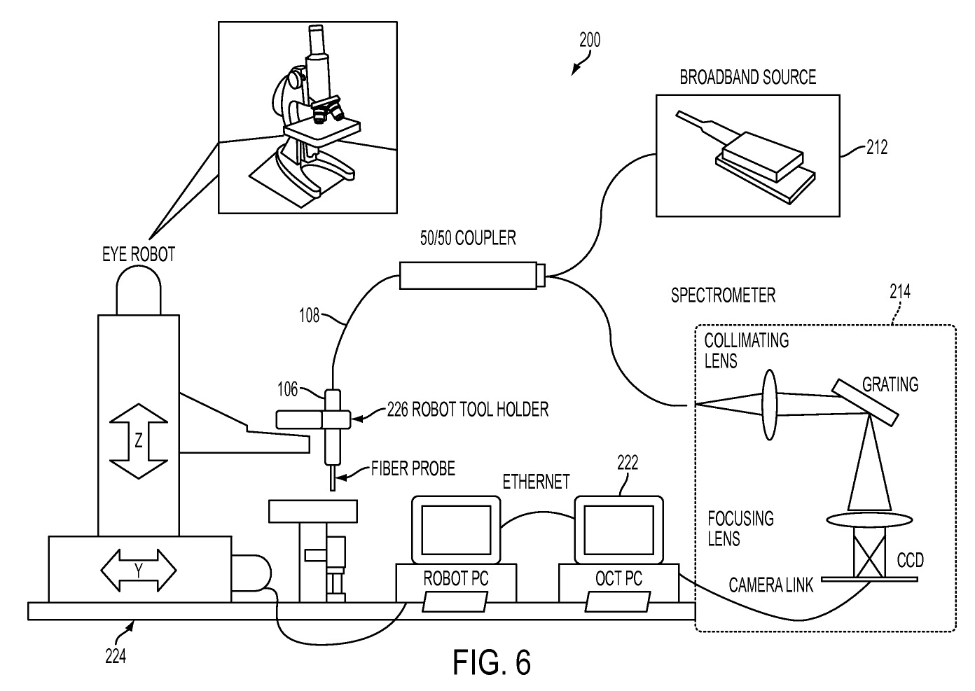

FIG. 6 provides a schematic illustration of an autofocusing endoscope 200 according to another embodiment of the current invention. The autofocusing endoscope 200 can have some components the same as, or similar to that of autofocusing endoscope 100. Although not visible in FIG. 6, the autofocusing endoscope 200 can have an objective lens 102, a relay optical system 104 arranged to relay an image between the objective lens 102 and a proximal end 106 of the autofocusing endoscope 200, an optical fiber 108 arranged with a distal end 110 proximate the objective lens 102, a light source 212 arranged to couple light into the optical fiber 108, and an optical detection system 214 arranged to receive and detect light from the optical fiber 108. The autofocusing endoscope 200 also has a data processor 222 constructed to communicate with the optical detection system 214 while in operation. The data processor 222 can be combined together with the optical detection system 214 or can be one or more separate components as illustrated in the example of FIG. 6. The data processor 222 is configured to determine a distance of a surface to be imaged through the objective lens 102 and provide instructions for adjusting a focus of the autofocusing endoscope 200 of the surface.

In the embodiment of FIG. 6, a robotic system 224 can be a portion of, or can be used as, an actuator assembly. For example, in one embodiment, autofocusing endoscope 200 can have an actuator assembly at tool holder 226, such as actuator assembly 118. The robotic system 224 can provide additional motion of translation and/or orientation, for example. In other embodiments, the robotic system 224 can provide the entire motion for the autofocusing, for example.

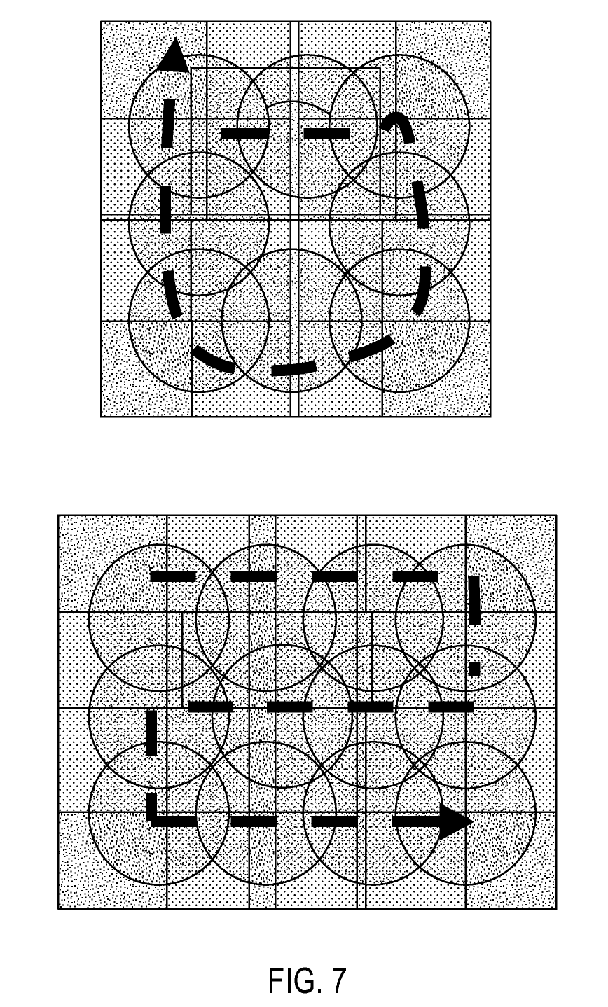

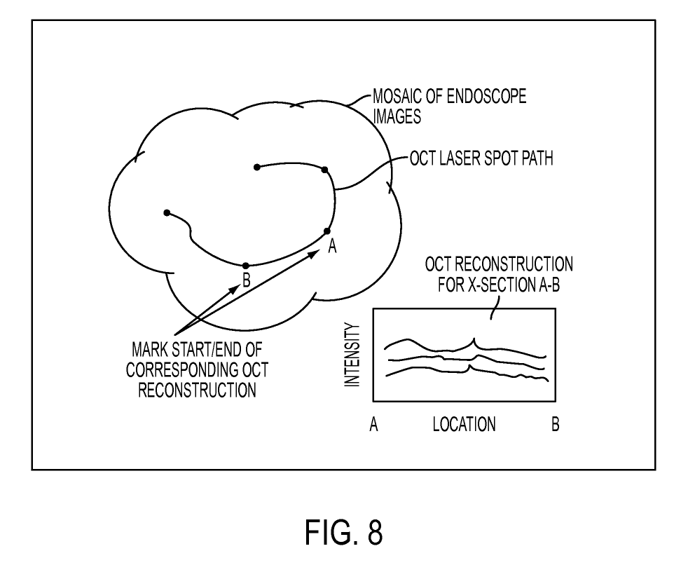

In some embodiments of the current invention, the data processor (e.g., but not limited to, data processor 222) can be further configured to provide instructions to the actuator assembly 118 and/or robotic system 224, for example, to scan the objective lens 102 and the distal end 110 of the optical fiber 108 to provide at least an image of a wider region of the surface while substantially maintaining focus during the scanning. In some embodiments, this can be an automatic scanning function. Autofocusing endoscopes according to some embodiments of the current invention can be scanned in a spiral and/or raster pattern, for example, as illustrated in FIG. 7. In some embodiments, a mosaic image as well as registered OCT images can be provided, as is illustrated in an example in FIG. 8.

The images provided by the moving endoscope can be registered together to “stitch” a mosaic image which has a larger field of view. This can be done by translating a handheld endoscope or used with a robotic assistant which autonomously or semi-autonomously translates the endoscope across a region of interest. In particular, we note two efficient strategies for imaging (a spiral, and a grid pattern) which allow for continuous smooth motion, and significant overlap, and are suitable for autonomous or semi-autonomous implementation. However, the general concepts of the current invention are not limited to this example. The mosaicked image is then used to determine the position of individual A-Scans to construct a cross-sectional image similar to a B-Scan. Likewise the projected laser spot can be segmented from endoscope image and used to estimate the spatial relationship between endoscope image frames. These transforms may be a homogenous transformation establishing a rigid relationship for very small areas, or a deformable map for larger regions.

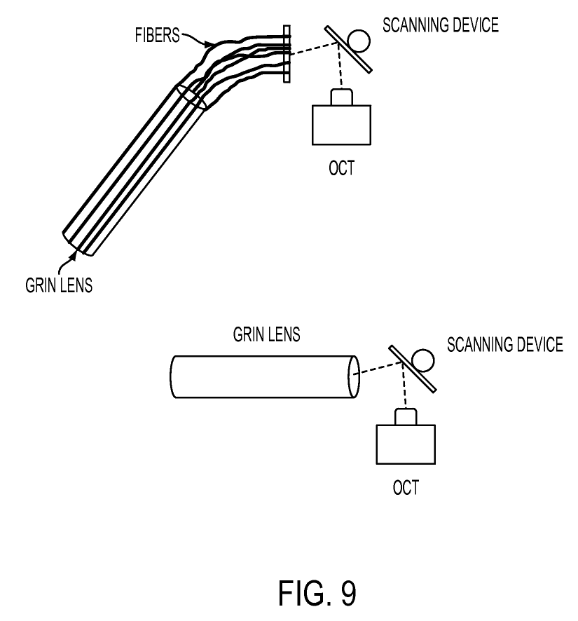

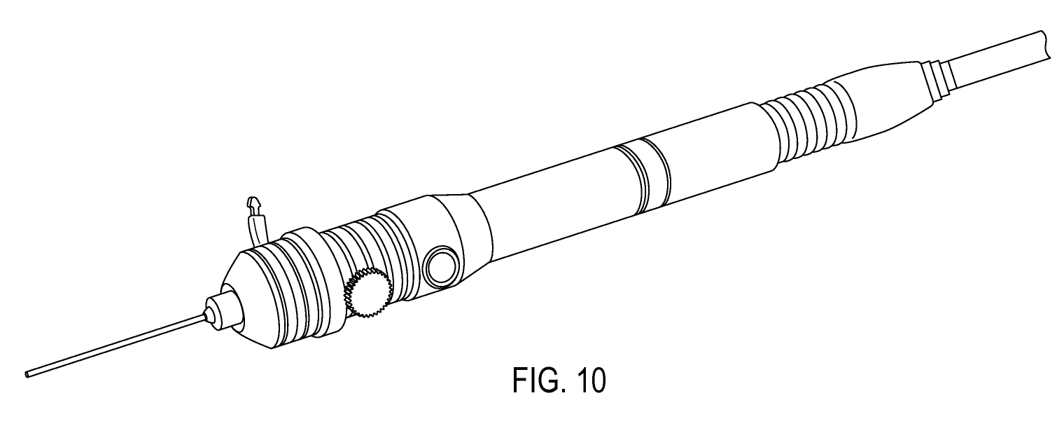

FIG. 9 illustrates further embodiments of the current invention in which each optical fiber of a plurality of optical fibers is arranged with a distal end proximate the objective lens, which is a GRIN lens in this example. In this embodiment a single OCT system can be used to selectively address each of the plurality of optical fibers or a separate OCT system can be integrated with each of the plurality of optical fibers, for example. These fibers may be scanned sequentially using, for example, but not limited to, a galvanometer-mirror arrangement. If only the distance to the surface is desired, then another interferometric range finding method (such as Fabry-Perot interferometry, for example) may be substituted for OCT.

In yet another embodiment, a scanning device may direct the OCT imaging path directly into the GRIN lens, if necessary using suitable auxiliary optics. In this case, the OCT can be used to produce both an en-face image, such as provided by a video camera and a c-mode OCT image of the targeted anatomy. Also, suitable optics and methods can be used to provide imaging paths through the lens both for a video camera for video endoscopy and for the OCT system.

FIG. 10 shows an example of a prototype of a hand-held autofocusing micro-endoscope according to an embodiment of the current invention.

Various embodiments of the current invention can provide, but are not limited to, one or more of the following:

1. Active focal distance control using OCT for feedback
2. Integrated OCT imaging with endoscope view
3. Integrated laser “spot” for OCT-Endoscope registration
4. Method for displaying Endoscope Imagery (mosaicing) with registered OCT data.
5. Provide precise and active illumination for endoscopic viewing
6. The active focal distance control where the probe is moved can also prevent direct collisions with the objects in front of the endoscope.
7. Efficient high resolution imaging of the retina where the OCT is used to maintain the constant distance from the retina, while the probe is moved to acquire high resolution visual images.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. An autofocusing endoscope, comprising:
an objective lens;
a relay optical system arranged to relay an image between said objective lens and a proximal end of said autofocusing endoscope;
an optical fiber arranged with a distal end proximate said objective lens;
a light source arranged to couple light into said optical fiber;
an optical detection system arranged to receive and detect light from said optical fiber; and
a data processor constructed to communicate with said optical detection system while in operation,
wherein said data processor is configured to determine a distance of a surface to be imaged through said objective lens and provide instructions for adjusting a focus of said autofocusing endoscope of said surface.

2. An autofocusing endoscope according to claim 1, further comprising an endoscope body and an actuator assembly attached to said endoscope body such that said actuator assembly moves at least said objective lens and said distal end of said optical fiber relative to said surface based on instructions received from said data processor to adjust said focus.

3. An autofocusing endoscope according to claim 1, wherein said objective lens is a gradient index (GRIN) objective lens.

4. An autofocusing endoscope according to claim 1, wherein said objective lens comprises at least one of a compound lens, a refractive lens, a diffractive lens, or a gradient index (GRIN) lens.

5. An autofocusing endoscope according to claim 1, wherein said relay optical system is a bundle of optical fibers.

6. An autofocusing endoscope according to claim 1, wherein said relay optical system is a lens system.

7. An autofocusing endoscope according to claim 5, wherein said optical fiber is combined into a bundle with said bundle of optical fibers of said relay optical system such that said optical fiber emits and receives light from said distal end through said objective lens.

8. An autofocusing endoscope according to claim 1, wherein said relay optical system comprises at least one of a refractive lens, a diffractive lens, a GRIN lens, an optical fiber, a light pipe, or an optical waveguide.

9. An autofocusing endoscope according to claim 1, wherein said optical fiber, said optical detection system, said light source, and said data processor together comprise an optical coherence tomography system.

10. An autofocusing endoscope according to claim 2, wherein said actuator assembly is at least part of a robotic system.

11. An autofocusing endoscope according to claim 2, wherein said actuator assembly is a compact actuator assembly such that said autofocusing endoscope is a hand-held autofocusing endoscope.

12. An autofocusing endoscope according to claim 1, further comprising an illumination light source optically coupled to said relay optical system to provide illumination light to illuminate said surface being imaged.

13. An autofocusing endoscope according to claim 2, further comprising an illumination light source optically coupled to said relay optical system to provide illumination light to illuminate said surface being imaged,
wherein said data processor is further configured to provide instructions to said actuator assembly to scan said objective lens and said distal end of said optical fiber to provide at least an image of a wider region of said surface while substantially maintaining focus during said scanning.

14. An autofocusing endoscope according to claim 13, wherein said optical fiber, said optical detection system, said light source, and said data processor together comprise an optical coherence tomography system, and
wherein said data processor registers optical coherence tomography images with said image of said wider region.

15. An autofocusing endoscope according to claim 1, further comprising a plurality of optical fibers each arranged with a distal end proximate said objective lens,
wherein said optical detection system is arranged to receive and detect light from each of said plurality of optical fibers, and
wherein said data processor is configured to determine a distance of a portion of said surface relative to each of said distal ends of said plurality of optical fibers and provide instructions for adjusting an orientation of said objective lens relative to said surface.
